(19) United States
(12) Patent Application Publication
(10) Pub. No.: US 2008/0027141 A1
(43) Pub. Date: Jan. 31, 2008

(54) 1,3-Dihydroxyl-9,10-anthraquinone and 3-[(3-amino)-propoxy]- 9,10-anthraquinone derivatives and pharmaceutical compositions comprising the same

(75) Inventors: Chun-Nan Lin, Kaohsiung (TW); Shen-Jeu Won, Tainan (TW); Chi-Hung Teng, Kaohsiung Hsien (TW)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Kaohsiung Medical University, Kaohsiung (TW)

(21) Appl. No.: 11/482,200

(22) Filed: Jul. 5, 2006

(51) Int. Cl. A61K 31/12; C07C 50/20

(52) U.S. Cl. 514/680; 552/245

(57) ABSTRACT

Disclosed herein are 1,3-dihydroxyl-9,10-anthraquinone and 3-[(3-amino)-propoxy]-9,10-anthraquinone derivatives, in particular 1,3-dihydroxy-4-prenyl-9,10-anthraquinone and 3-[3-(4-methylpiperazinyl)-propoxy]-9,10-anthraquinone, which are found to have inhibitory activities against several types of human tumor/cancer cells and thus can be used in the manufacture of pharmaceutical compositions for use in the treatment of tumors/cancers.

### BACKGROUND OF THE INVENTION

1. Field of the Invention

This invention relates to 1,3-dihydroxyl-9,10-anthraquinone and 3-[(3-amino)-propoxy]-9,10-anthraquinone derivatives, in particular 1,3-dihydroxy-4-prenyl-9,10-anthraquinone and 3-[3-(4-methylpiperazinyl)-propoxy]-9,10-anthraquinone, which are found to have inhibitory activities against several types of human tumor/cancer cells. This invention also relates to the uses of said compounds in the manufacture of pharmaceutical compositions for use in the treatment of tumors/cancers.

2. Description of the Related Art

Apoptosis is considered to be the major process responsible for cell death in various physiological events, and it acts as a regulating mechanism of tissue growth, where it balances cell proliferation (Kerr, J F R (1971), J. Pathol., 105: 13-20). When human cells are damaged and cannot be repaired, apoptosis will be initiated so as to avoid the formation of cancer cells. The major morphological features of apoptosis include: formation of apoptotic bodies, chromatin condensation, and DNA fragmentation (Arends, M. J. and Wyllie, A. H. (1991), Int. Rev. Exp. Pathol., 32: 223-254; Dive, C., et al. (1992), Biochim. Biophys. Acta, 1133: 275-285; and Darzynkiewicz, Z., et al. (1992), Cytometry, 13: 795-808). During apoptosis, debris of dead cells will be rapidly ingested by neighboring cells and macrophages via phagocytosis without inducing an inflammatory response (Sarraf, F. E. and Bowen, I. D. (1988), Cell Tissue Res., 21: 45-49). In addition, when the variation of cell cycle is detected by flow cytometry, the presence of a sub-G1 peak can be observed (Alzerreca, A. and Hart, G. (1982), Toxicology Lett., 12: 151-155; and Lin, C. N., et al. (1986), J. Taiwan Pharm. Assoc. 38, 166). Thus, the sub-G1 peak is considered to be a typical marker for identifying cells that are undergoing apoptosis.

It is reported in literature that cells will become cancer cells if the apoptotic mechanism thereof is out of control (Carson, D. A. and Ribeiro, J. M. (1993), Lancet , 341: 1251-1254; and Kaufmann, S. H. (1989), Cancer Res., 49: 5870-5878). Therefore, apoptosis has become a subject of study in oncology. In addition, it is reported that apoptosis can be induced by certain anti-cancer drugs (Wyllie, A. H., et al. (1980), Int Rev. Cytol., 68: 251-306; Wyllie, A. H. et al. (1984), J Pathol., 142: 67-77; Barry, M. A. et al. (1990), Biochem. Pharmacol., 40: 2353-2362; and Hickman, J. A. (1992), Cancer Metast. Rev., 11: 121-139). Thus, apoptosis points to a major direction in the global development of anti-cancer drugs.

In the earlier studies of the applicants, a series of 1-hydroxy-3-(ω-alkylamino-propoxy)-9,10-anthraquinone derivatives were synthesized, which were tested to exert potent cytotoxic effects (Wei, Bai-Luh et al. (2000), Eur. J. Med. Chem., 35: 1089-1098). In an effort to develop new potent anti-tumor agents, which may lead to tumor cell apoptosis, the Applicants further synthesized a different series of compounds, including 1,3-dihydroxy-4-prenyl-9,10-anthraquinone and 3-[3-(4-methylpiperazinyl)-propoxy]-9,10-anthraquinone, and evaluated their inhibitory effect upon human tumor/cancer cells.

### SUMMARY OF THE INVENTION

According to a first aspect, this invention provides a compound of formula (A):

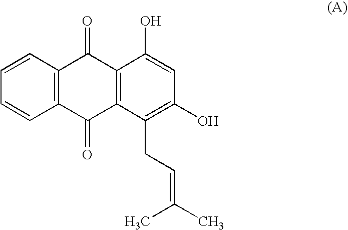

or a pharmaceutically acceptable salt thereof.

In a second aspect, this invention provides a compound of formula (B):

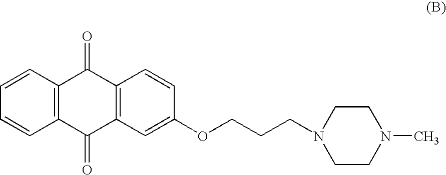

or a pharmaceutically acceptable salt thereof.

The aforesaid compounds were found to have cytotoxicity against tumor/cancer cells. Therefore, in a third aspect, this invention provides a pharmaceutical composition comprising as an active ingredient a compound of formula (A) or formula (B), or a pharmaceutically acceptable salt thereof, and optionally a pharmaceutically acceptable carrier.

In addition, in a fourth aspect, the present invention provides a method for inhibiting the growth of tumor/cancer cells in a subject, comprising administering to the subject a therapeutically effective amount of a compound of formula (A) or formula (B), or a pharmaceutically acceptable salt thereof.

### BRIEF DESCRIPTION OF THE DRAWINGS

The above and other features and advantages of the present invention will become apparent in the following detailed description of the preferred embodiments with reference to the accompanying drawing, of which:

FIG. 1 shows the results of flow cytometry analysis of Compound 16 according to this invention, in which MCF-7 cells (1×10⁴ cells/mL) were treated with medium only (control group) or various concentrations (6.9, 13.7, and 27.5 μM) of Compound 16 for different time periods (24 h, 48 h and 72 h), followed by staining with propidium iodide (PI). DNA contents were analyzed by flow cytometry, and apoptosis was measured by the accumulation of sub-G1 DNA contents in cells. Results were representative of three independent experiments; and

FIG. 2 shows the results of DNA fragmentation in MCF-7 cells (1×10⁴ cells/mL) treated with various concentrations (6.9, 13.7, and 27.5 μM) of Compound 16 according to this invention for different time periods (24 h, 48 h and 72 h), as analyzed by 1% agarose gel electrophoresis, in which panel (A): 24 hours incubation; panel (B): 48 hours incubation; panel (C): 72 hours incubation; lane M: 100 bp marker (Lambda Biotech); lane C: control group treated with medium only; lane 1: cells treated with 6.9 μM of Compound 16, lane 2: cells treated with 13.7 μM of Compound 16; and lane 3: cells treated with 27.5 μM of Compound 16. Results were representative of three independent experiments.

### DETAILED DESCRIPTION OF THE INVENTION

For the purpose of this specification, it will be clearly understood that the word “comprising” means “including but not limited to,” and that the word “comprises” has a corresponding meaning.

It is to be understood that, if any prior art publication is referred to herein, such reference does not constitute an admission that the publication forms a part of the common general knowledge in the art in any country.

Unless otherwise defined, all technical and scientific terms used herein have the meaning commonly understood by a person skilled in the art to which this invention belongs.

In the earlier studies of the applicants, a series of 1-hydroxy-3-(ω-alkylamino-propoxy)-9,10-anthraquinone derivatives were synthesized, which were tested to exert potent cytotoxic effects (Wei, Bai-Luh et al. (2000), Eur. J. Med. Chem., 35: 1089-1098). To continuously develop new potent anti-tumor agents, which may lead to tumor cell apoptosis, the applicants further designed and synthesized another series of 1,3-dihydroxy-9,10-anthraquinone (DHA), 1-hydroxy-3-[(3-amino)-propoxy]-9,10-anthraquinone (MHA) and 3-[(3-amino)-propoxy]-9,10-anthraquinone (NHA) derivatives.

According to this invention, these new derivatives may be synthesized by methods described previously (see, e.g., Wei, Bai-Luh et al. (2000), supra). Briefly, referring to the General Synthesis Scheme shown below, potassium salt of 1,3-dihydroxy-9,10-anthraquinone (DHA) (Compound 1) or 3-hydroxy-9,10-anthraquinone (Compound 3) was allowed to react with 1,3-dibromopropane in an appropriate solvent, followed by amination with appropriate amines, thereby giving various 1-hydroxy-3-[(3-amino)-propoxy]-9,10-anthraquinones and 3-[(3-amino)-propoxy])-9,10-anthraquinones. On the other hand, Compound 5 may be produced from the reaction of Compound 1 in MeOH with prenyl bromide in the presence of NaOMe.

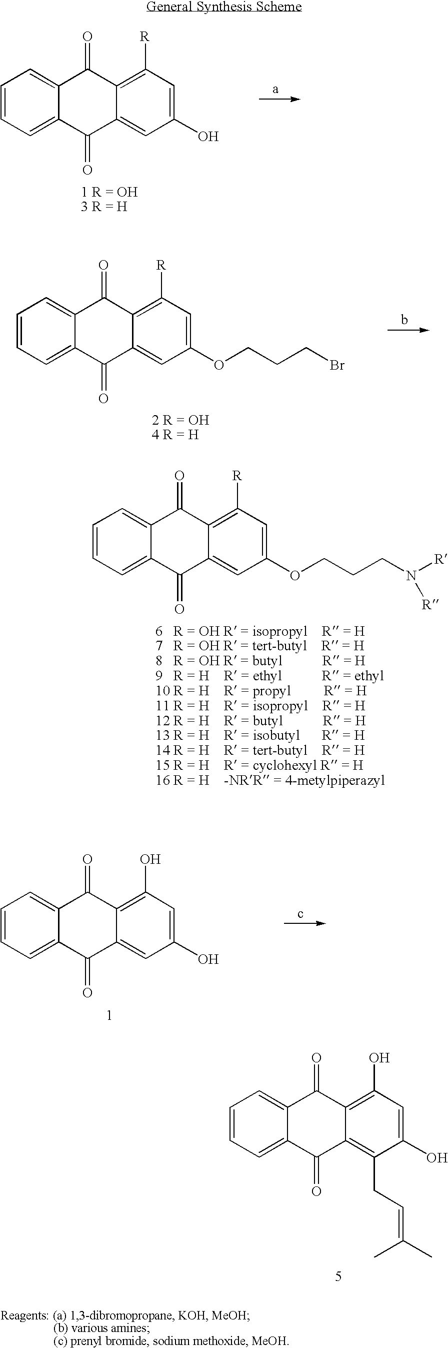

These 9,10-anthraquinone derivatives were tested to determine the cytotoxicity activity thereof against human tumor/cancer cells, amongst which 1,3-dihydroxy-4-prenyl-9,10-anthraquinone (Compound 5 of synthesis example 1, infra) selectively enhanced the cytotoxic effects against HepG2 and Hep3B cells in vitro, and 3-[3-(4-methylpiperazinyl)-propoxy]-9,10-anthraquinone (Compound 16 of synthesis example 12, infra) shows potent cytotoxic activity against HT-29 and MCF-7 cells.

Therefore, this invention provides a compound of formula (A):

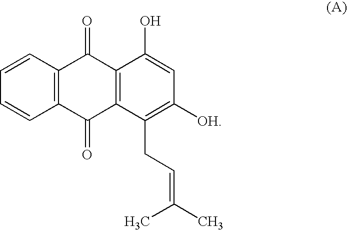

This invention also provides a compound of formula (B):

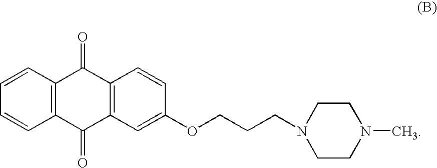

The above two compounds according to this invention may be in their free form or in the form of a pharmaceutically acceptable salt thereof.

Illustrative pharmaceutically acceptable salts include metal salts such as sodium salt, potassium salt, calcium salt, magnesium salt, manganese salt, iron salt and aluminum salt; mineral acid addition salts such as hydrochloride, hydrobromide, hydroiodide, sulfate and phosphate.

In addition, the compounds of this invention may also exist as a stereoisomer or in the form of solvates represented by the hydrate. Therefore, it is contemplated that these stereoisomers and solvates fall within the technical concept of this invention.

The compounds of this invention have been demonstrated to exhibit inhibitory activities against the growth of several types of tumor/cancer cells, in particular human liver cancer cells (such as HepG2 and Hep3B), human colon/rectal cancer cells (such as HT-29), and human breast cancer cells (such as MCF-7). Therefore, the present invention envisions the application of said compounds in the manufacture of pharmaceutical compositions for use in tumor/cancer therapy.

Optionally, the pharmaceutical composition according to this invention may additionally contain a pharmaceutically acceptable carrier commonly used in the art for the manufacture of medicaments. For example, the pharmaceutically acceptable carrier can include one or more than one of the following reagents: solvents, disintegrating agents, binders, excipients, lubricants, absorption delaying agents and the like.

The pharmaceutical composition according to this invention may be administered parenterally or orally in a suitable pharmaceutical form. Suitable pharmaceutical forms include sterile aqueous solutions or dispersions, sterile powders, tablets, troches, pills, capsules, and the like.

The unit dosage form of the pharmaceutical compositions may, in accordance with the object of a therapy, be suitably chosen from any one of oral preparations, injections, suppositories, ointments, inhalants, eye drops, nasal drops, plasters and the like. These unit dosage forms can each be prepared by a preparation method commonly known and used by those skilled in the art.

To produce an oral solid preparation, an excipient and, if necessary, a binder, a disintegrator, a lubricant, a coloring matter, a flavoring agent and/or the like may be admixed with a compound of this invention. The resultant mixture can then be formed into tablets, coated tablets, granules, powder, capsules or the like by a method known per se in the art. Such additives can be those generally employed in the present field of art, including excipients: lactose, sucrose, sodium chloride, glucose, starch, calcium carbonate, kaolin, micro-crystalline cellulose, and silicic acid; binders: water, ethanol, propanol, sucrose solution, glucose solution, starch solution, gelatin solution, carboxymethylcellulose, hydroxypropylcellulose, hydroxypropylstarch, methylcellulose, ethylcellulose, shellac, calcium phosphate, and polyvinylpyrrolidone; disintegrators: dry starch, sodium alginate, powdered agar, sodium hydrogencarbonate, calcium carbonate, sodium lauryl sulfate, monoglycerol stearate, and lactose; lubricants: purified talc, stearate salts, borax, and polyethylene glycol; and corrigents: sucrose, bitter orange peel, citric acid, and tartaric acid.

To produce an oral liquid preparation, a flavoring agent, a buffer, a stabilizer and the like may be admixed with a compound of this invention. The resultant mixture can then be formed into a solution for internal use, a syrup, an elixir or the like by a method known per se in the art. In this case, the flavoring agent can be the same as that mentioned above. Illustrative of the buffer is sodium citrate, while illustrative of the stabilizer are tragacanth, gum arabic, and gelatin.

To prepare an injection, a pH regulator, a buffer, a stabilizer, an isotonicity and the like may be admixed with a compound of this invention. The resultant mixture can then be formed into a subcutaneous, intramuscular or intravenous injection by a method known per se in the art. Examples of the pH regulator and buffer include sodium citrate, sodium acetate, and sodium phosphate. Illustrative of the stabilizer include sodium pyrosulfite, EDTA, thioglycollic acid, and thiolactic acid. Examples of the isotonicity include sodium chloride and glucose.

To prepare suppositories, a pharmaceutical carrier known in the present field of art, for example, polyethylene glycol, lanolin, cacao butter or fatty acid triglyceride may be added, optionally together with a surfactant such as “Tween” (registered trademark), to a compound of the present invention. The resultant mixture can then be formed into suppositories by a method known per se in the art.

To prepare an ointment, a pharmaceutical base, a stabilizer, a humectant, a preservative and the like are combined, as needed, with a compound of this invention. The resultant mixture can then be mixed and prepared into an ointment by a method known per se in the art. Illustrative of the pharmaceutical base are liquid paraffin, white petrolatum, white beeswax, octyldodecyl alcohol, and paraffin. Examples of the preservative include methyl parahydroxybenzoate, ethyl parahydroxybenzoate, and propyl parahydroxybenzoate.

In addition to the above-described preparations, the compounds of the present invention may also be formed into an inhalant or a nasal drop by methods known per se in the art.

The term “therapeutically effective amount” as used herein refers to an amount of the pharmaceutical composition according to this invention which is sufficient to provide a desired therapeutic effect when administered to a subject in need of such treatment without causing undesired damage to the non-targeted tissues or organs of said subject.

Dosage amount and interval of the pharmaceutical composition according to this invention are dependent upon the following factors: severity of the disease to be treated, administering route, and the weight, age, health condition and response of the subject to be treated.

Optionally, the pharmaceutical composition according to the present invention can be administered singly, or in combination with other therapeutic methods or therapeutic medicaments for use in the treatment of tumors or cancers. Such therapeutic methods include chemotherapy and external beam radiation therapy. Such therapeutic medicaments include, but are not limited to, 5-fluorouracil (5-FU), paclitaxel, mytomycin, cyclophosphamide, adriamycin, doxorubicin, actinomycin, cisplatin, carboplatin, and the like.

### EXAMPLES

The present invention will now be described in more detail with reference to the following examples, which are given for the purpose of illustration only and are not intended to limit the scope of the present invention.

### General Procedures:

Melting points (uncorrected) were determined using a Yanco Micro-Melting point apparatus. IR spectra were determined using a Perkin-Elmer system 2000 FTIR spectrophotometer. ¹H (400 MHz) and ¹³C (100 MHz) NMR were recorded on a Varian UNITY-400 spectrometer, and mass were obtained on a JMX-HX 100 mass spectrometer. Elemental analyses were within ±0.4% of the theoretical values, unless otherwise noted. Chromatography was performed using a flash-column technique on silica gel 60 supplied by E. Merck.

1,3-dihydroxy-9,10-anthraquinone (Compound 1), 1-hydroxy-3-(3-bromopropoxy)-9,10-anthraquinone (Compound 2), 3-hydroxy-9,10-anthraquinone (Compound 3) and 3-(3-bromopropoxy)-9,10-anthraquinone (Compound 4) used in the following synthesis examples were prepared according to known methods (see, e.g., Wei, Bai-Luh et al. (2000), supra).

### Synthesis Ex. 1

### 1,3-Dihydroxy-4-prenyl-9,10-anthraquinone (Compoud 5)

1,3-dihydroxy-9,10-anthraquinone (0.482 g, 2 mmol) (Compound 1) in anhydrous methanol was added to methanolic solution of sodium methoxide (9 mL). Prenyl bromide (2 mL) was added to the mixture under ice bath, and the mixture was then refluxed for 3 h. After removal of the solvent, water was added to the mixture, and the mixture was acidified with concentrated HCl. The precipitated solid was collected and purified by chromatography (silica gel and n-hexane-EtOAc (4:1)), giving the title compound as orange needles (CHCl₃) (0.027 g, 0.08 mmol, 4.0%).

### Detected Properties of the Title Compound:

Mp: 198° C.

IR (KBr) 3391, 1665, 1624, 1590 cm⁻¹.

¹H NMR ((CD₃)₂CO): δ 1.67 (3H, s, Me), 1.80 (3H, s, Me), 3.45 (2H, d, J=7.3 Hz, —CH₂CH═), 5.28 (1H, m,

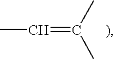

### 7.35 (1H, s, H-2), 7.86-7.92 (2H, m, H-6 and H-7), 8.17-8.30 (2H, m, H-5 and H-8), 13.23 (1H, s, OH-1).

¹³C NMR ((CD₃)₂CO): δ 18.7 (Me), 23.6 (Me), 26.6 (—CH₂CH═), 108.4 (C-9a), 109.1 (C-2), 122.6

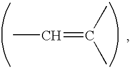

### 123.2 (C-4), 128.1 (C-8) 128.3 (C-5), 133.5 (C-4a), 134.2 (C-10a), 135.1 (C-8a), 135.2

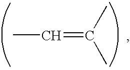

### 135.8 (C-6 and C-7), 163.7 (C-3), 164.6 (C-1), 183.3 (C-10), 188.6 (C-9).

EIMS (70 eV) m/z (% rel int.): 308 (56) [M]⁺.

Anal Calcd for C₁₇H₁₆O₄: C, 74.00; H, 5.20. Found: C, 73.50; H, 5.26.

### Synthesis Ex. 2

### 1-Hydroxy-3-[3-(isopropylamino)-propoxy]-9,10-anthraquinone (Compound 6)

Compound 2 (0.12 g, 0.33 mmol) in EtOH (40 mL) was admixed with propylamine (1.37 g, 23.10 mmol) and then refluxed for 1 h. The resultant product was purified by column chromatography (silica gel and MeOH) and crystallized from MeOH, giving the title compound as green needles (0.018 g, 0.05 mmol, 18.7%).

### Detected Properties of the Title Compound:

Mp: 212° C.

IR (KBr) 3421, 1670, 1635, 1592 cm⁻¹.

¹H NMR (DMSO-d₆): δ 1.28 (6H, d, J=6.8 Hz, 2×Me), 2.18 (2H, m, —OCH₂CH₂—), 3.06 (2H, m, —CH₂NH—), 3.25 (1H, m, —CH(CH₃)₂), 4.28 (2H, t, J=6 Hz, —OCH₂—), 6.88 (1H, d, J=2.4 Hz, H-2), 7.19 (1H, d, J=2.4 Hz, H-4), 7.92 (2H, m, H-6 and H-7), 8.18 (2H, m, H-5 and H-8), 9.11 (2H, br s, —N^{⊖}H₂—), 12.76 (1H, s, OH-1).

¹³C NMR (CDCl₃): δ 18.5 (2×Me), 25.3 (—OCH₂CH₂—), 40.9 (—CH₂NH—), 49.6

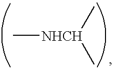

66.0 (—OCH₂—), 106.7 (C-2), 107.6 (C-4), 110.3 (C-9a), 126.4 (C-8), 126.8 (C-5), 132.9 (C-8a and C-10a), 134.7 (C-6 and C-7), 134.9 (C-4a), 164.6 (C-1), 164.9 (C-3), 181.6 (C-10), 186.3 (C-9).

EIMS (70 eV) m/z (% rel int.): 339 (4) [M]⁺.

HREIMS m/z [M]⁺ 339.1468 (calcd for C₂₀H₂₁NO₄, 339.1470).

### Synthesis Ex. 3

### 3-[3-(tert-Butylamino)-propoxy]-1-hydroxy-9,10-anthraquinone (Compound 7)

The title compound was obtained as yellow needles (0.016 mg, 0.045 mmol, 16.2%) according to the procedures set forth in the above Synthesis Example 2, except that tert-butylamine (1.78 g, 24.30 mmol) was used in place of propylamine.

### Detected Properties of the Title Compound:

Mp: 205° C.

IR(KBr) 3447, 1670, 1634, 1593 cm⁻¹.

¹H NMR (DMSO-d₆): δ 1.32 (9H, s, Me), 2.17 (2H, m, —OCH₂CH₂—), 3.04 (2H, br s, —CH₂NH—), 4.31 (2H, t, J=6.2 Hz, —OCH₂—), 6.92 (1H, d, J=2.4 Hz, H-2), 7.24 (1H, d, J=2.4 Hz, H-4), 7.94 (2H, m, H-6 and H-7), 8.21 (2H, m, H-5 and H-8), 9.09 (2H, br s, —N^{⊖}H₂—) 12.76 (1H, s, OH-1).

¹³C NMR (DMSO-d₆): δ 25.1 (Me), 25.8 (—OCH₂CH₂—), 37.7 (—NHC—), 55.9 (—CH₂NH—), 66.1 (—OCH₂—), 106.8 (C-2), 107.6 (C-4), 110.3 (C-9a), 126.4 (C-8), 126.9 (C-5), 132.9 (C-8a and C-10a), 134.7 (C-6, C-7 and C-4a), 164.5 (C-1), 165.0 (C-3), 181.6 (C-10), 186.3 (C-9).

EIMS (70 eV) m/z (% rel int.): 353 (1) [M]⁺.

HREIMS m/z [M]⁺ 353.1630 (calcd for C₂₁H₂₃NO₄, 353.1627).

### Synthesis Ex. 4

### 3-[3-(Butylamino)-propoxy]-1-hydroxy-9,10-anthraquinone (Compound 8)

The title compound was obtained as yellow needles (0.030 mg, 0.084 mmol, 30.3%) according to the procedures set forth in the above Synthesis Example 2, except that n-butylamine (1.61 g, 22.0 mmol) was used in place of propylamine.

### Detected Properties of the Title Compound:

Mp: 220° C.

IR (KBr) 3402, 1670, 1636, 1592 cm⁻¹.

¹H NMR (DMSO-d₆): δ 0.90 (3H, t, J=2.6, Me), 1.34 (2H, m, —CH₂CH₃), 1.62 (2H, m, —CH₂CH₂CH₃), 2.14 (2H, m, —OCH₂CH₂—), 2.51 (2H, br s, —CH₂NHCH₂—), 3.08 (2H, br s, —CH₂NHCH₂—), 4.28 (2H, t, J=6.0 Hz, —OCH₂—), 6.89 (1H, d, J=2.6 Hz, H-2), 7.21 (1H, d, J=2.4 Hz, H-4), 7.99 (2H, m, H-6 and H-7), 8.18 (2H, m, H-5 and H-8), 8.93 (2H, br s, —N^{⊖}H₂—), 12.76 (1H, s, OH-1).

¹³C NMR (DMSO-d₆): δ 13.4 (Me), 19.3 (—CH₂CH₃), 25.1 (—CH₂CH₂CH₃), 27.5 (—OCH₂CH₂—), 43.9 (—CH₂NH—), 46.6 (—NHCH₂—), 66.0 (—OCH₂—), 106.7 (C-2), 107.6 (C-4), 110.3 (C-9a), 126.4 (C-8), 126.9 (C-5), 132.9 (C-8a and 10a), 134.7 (C-6, C-7 and C-4a), 164.6 (C-1), 164.9 (C-3), 181.6 (C-10), 186.3 (C-9).

EIMS (70 eV) m/z (% rel int.): 353 (1) [M]⁺.

HREIMS m/z [M]⁺ 353.1618 (calcd for C₂₁H₂₃NO₄, 353.1627).

### Synthesis Ex. 5

### 3-[3-(Diethylamino)-propoxy]-9,10-anthraquinone (Compound 9)

Compound 4 (0.1 g, 0.28 mmol) in EtOH (40 mL) was added with diethylamine (1.42 g, 19.39 mmol) and then refluxed for 1 h. The resultant product was purified by column chromatography (silica gel and MeOH) and crystallized from MeOH, giving the title compound as a yellow powder (0.031 g, 0.09 mmol, 32.1%).

### Detected Properties of the Title Compound:

IR (KBr) 1670, 1590 cm⁻¹.

¹H NMR (DMSO-d₆): δ 1.23 (6H, t, J=7.4 Hz, 2×Me), 2.19 (2H, m, —OCH₂CH₂—), 3.21 (6H, m, —CH₂N(CH₂)₂—), 4.32 (2H, t, J=5.6 Hz, —OCH₂—), 7.47 (1H, dd, J=7.8, 2.8 Hz, H-2), 7.64 (1H, d, J=2.4 Hz, H-4), 7.94 (2H, m, H-6 and H-7), 8.20 (3H, m, H-1, H-5 and H-8), 9.80 (1H, br s,

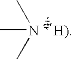

¹³C NMR (DMSO-d₆): δ 8.5 (Me), 23.0 (—OCH₂CH₂—), 46.3 (—N(CH₂)₂—), 47.7 (—CH₂NH—), 65.7 (—OCH₂—), 110.8 (C-4), 121.0 (C-2), 126.5 (C-9a), 126.6 (C-8), 126.7 (C-5), 129.5 (C-1), 133.0 (C-10a), 133.0 (C-8a), 134.2 (C-7), 134.6 (C-6), 135.0 (C-4a), 162.9 (C-3), 181.3 (C-9), 182.3 (C-10).

EIMS (70 eV) m/z (% rel int.): 337 (1) [M]⁺.

HREIMS m/z [M]⁺ 337.1679 (calcd for C₂₁H₂₃NO₃, 337.1678).

### Synthesis Ex. 6

### 3-[3-(Propylamino)-propoxy]-9,10-anthraquinone (Compound 10)

The title compound was obtained as a purplish powder (0.028 mg, 0.088 mmol, 30.2%) according to the procedures set forth in the above Synthesis Example 5, except that propylamine (0.79 g, 19.39 mmol) was used in place of diethylamine.

### Detected Properties of the Title Compound:

IR (KBr) 1667, 1589 cm⁻¹.

¹H NMR (CD₃OD): δ 1.05 (3H, t, J=7.6 Hz, Me), 1.75 (2H, m, —CH₂CH₃), 2.27 (2H, m, —OCH₂CH₂—), 3.03 (2H, m, —CH₂N—), 3.27 (2H, t, J=6 Hz, —NCH₂—), 4.34 (2H, t, J=5.6 Hz, —OCH₂—), 7.41 (1H, dd, J=8.8, 2.8 Hz, H-2), 7.76 (1H, d, J=2.4 Hz, H-4), 7.87 (2H, m, H-6 and H-7), 8.27 (3H, m, H-1, H-5 and H-8).

¹³C NMR (CD₃OD): δ 11.2 (Me), 20.8 (—NCH₂CH₂—), 27.2 (—OCH₂CH₂—), 46.5 (—NHCH₂—), 50.8 (—CH₂NH—), 66.9 (—OCH₂—), 112.0 (C-4), 122.1 (C-2), 128.0 (C-5 and C-8), 128.6 (C-9a), 130.8 (C-1), 134.9 (C-8a and C-10a), 135.1 (C-7), 135.6 (C-6), 136.9 (C-4a), 164.8 (C-3), 183.3 (C-9), 184.2 (C-10).

EIMS (70 eV) m/z (% rel int.): 323 (5) [M]⁺.

HREIMS m/z [M]⁺ 323.1517 (calcd for C₂₀H₂₁NO₃, 323.1521).

### Synthesis Ex. 7

### 3-[3-(Isopropylamino)-propoxy]-9,10-anthraquinone (Compound 11)

The title compound was obtained as a pink powder (0.027 mg, 0.083 mmol, 28.7%) according to the procedures set forth in the above Synthesis Example 5, except that isopropylamine (0.79 g, 19.39 mmol) was used in place of diethylamine.

### Detected Properties of the Title Compound:

IR (KBr) 1672, 1590 cm⁻¹.

¹H NMR (CD₃OD): δ 1.38 (6H, d, J=6.8 Hz, 2×Me), 2.27 (2H, m, —OCH₂CH₂—), 3.29 (2H, m, —CH₂N—), 3.45 (1H, m, —NCH—), 4.34 (2H, t, J=5.6 Hz, —OCH₂—), 7.40 (1H, dd, J=8.8, 2.8 Hz, H-2), 7.73 (1H, d, J=2.4 Hz, H-4), 7.86 (2H, m, H-6 and H-7), 8.25 (3H, m, H-1, H-5 and H-8).

¹³C NMR (CD₃OD): δ 19.3 (Me), 27.3 (—OCH₂CH₂—), 43.6 (—CH₂NH—), 52.1 (—NHCH—), 66.8 (—OCH₂—), 112.0 (C-4), 122.1 (C-2), 128.0 (C-5 and C-8), 128.6 (C-9a), 130.8 (C-1), 134.9 (C-8a and C-10a), 135.1 (C-7), 135.6 (C-6), 136.9 (C-4a), 164.8 (C-3), 183.2 (C-9), 184.1 (C-10).

EIMS (70 eV) m/z (% rel int.): 323 (7) [M]⁺.

HREIMS m/z [M]⁺ 323.1526 (calcd for C₂₀H₂₁NO₃, 323.1521).

### Synthesis Ex. 8

### 3-[3-(Butylamino)-propoxy]-9,10-anthraquinone (Compound 12)

The title compound was obtained as a yellowish powder (0.030 mg, 0.088 mmol, 30.4%) according to the procedures set forth in the above Synthesis Example 5, except that n-butylamine (0.98 g, 19.39 mmol) was used in place of diethylamine.

### Detected Properties of the Title Compound:

IR (KBr) 1671, 1591 cm⁻¹.

¹H NMR (DMSO-d₆): δ 0.90 (3H, t, J=7.2 Hz, Me), 1.35 (2H, m, —CH₂CH₃), 1.62 (2H, m, —CH₂CH₂CH₃), 2.20 (2H, m, —OCH₂CH₂—), 2.90 (2H, br s, —CH₂NH—), 3.09 (2H, br s, —NHCH₂—), 4.32 (2H, m, —OCH₂—), 7.45 (1H, dd, J=8.8, 2.8 Hz, H-2), 7.61 (1H, d, J=2.4 Hz, H-4), 7.92 (2H, m, H-6 and H-7), 8.19 (m, 3H, H-1, H-5 and H-8), 9.05 (2H, br s,

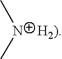

¹³C NMR (DMSO-d₆): δ 13.0 (Me), 18.8 (—CH₂CH₃), 24.7 (—NCH₂CH₂—), 26.9 (—OCH₂CH₂—), 43.3 (—CH₂NH—), 46.0 (—NHCH₂—), 65.2 (—OCH₂—), 110.3 (C-4), 120.6 (C-2), 126.0 (C-8), 126.1 (C-5), 126.2 (C-9a), 129.0 (C-1), 132.5 (C-10a), 132.5 (C-8a), 133.7 (C-7), 134.1 (C-6), 134.5 (C-4a), 162.6 (C-3), 180.8 (C-9), 181.9 (C-10).

EIMS (70 eV) m/z (% rel int.): 337 (1) [M]⁺.

HREIMS m/z [M]⁺ 337.1678 (calcd for C₂₁H₂₃NO₃, 337.1678).

### Synthesis Ex. 9

### 3-[3-(Isobutylamino)-propoxy]-9,10-anthraquinone (Compound 13)

The title compound was obtained as a pink powder (0.030 mg, 0.09 mmol, 31.1%) according to the procedures set forth in the above Synthesis Example 5, except that isobutylamine (0.98 g, 19.39 mmol) was used in place of diethylamine.

### Detected Properties of the Title Compound:

IR (KBr) 1673, 1593 cm⁻¹.

¹H NMR (DMSO-d₆): δ 1.07 (6H, d, J=8 Hz, 2×CH₃), 2.05 (1H, m, —CH(CH₃)₂), 2.30 (2H, m, —OCH₂CH₂—), 2.93 (2H, d, J=7.2 Hz, —NHCH₂—), 3.26 (2H, m, —NHCH₂—), 4.34 (2H, t, J=5.6 Hz, —OCH₂—), 7.40 (1H, dd, J=8.8, 2.8 Hz, H-2), 7.75 (1H, d, J=2.4 Hz, H-4), 7.87 (2H, m, H-6 and H-7), 8.26 (3H, m, H-1, H-5 and H-8).

¹³C NMR (DMSO-d₆): δ 20.3 (Me), 26.9 (—OCH₂CH₂—), 27.4 (—CH(CH₃)₂), 47.0 (—CH₂NH—), 56.3 (—NHCH₂—), 66.9 (—OCH₂—), 112.0 (C-4), 122.0 (C-2), 128.0 (C-5 and c-8), 128.6 (C-9a), 130.8 (C-1), 135.0 (C-8a and C-10a), 135.1 (C-7), 135.6 (C-6), 136.9 (C-4a), 164.8 (C-3), 183.3 (C-9), 184.2 (C-10).

EIMS (70 eV) m/z (% rel int.): 337 (1) [M]⁺.

HREIMS m/z [M]⁺ 337.1677 (calcd for C₂₁H₂₃NO₃, 337.1678).

### Synthesis Ex. 10

### 3-[3-tert-Butylamino)-propoxy]-9,10-anthraquinone (Compound 14)

The title compound was obtained as a light brown powder (0.033 mg, 0.097 mmol, 33.3%) according to the procedures set forth in the above Synthesis Example 5, except that tert-butylamine (0.98 g, 19.39 mmol) was used in place of diethylamine.

### Detected Properties of the Title Compound:

IR (KBr) 1673, 1591 cm⁻¹.

¹H NMR (CD₃OD): δ 1.41 (9H, s, 3×Me), 2.26 (2H, m, —OCH₂CH₂—), 3.24 (2H, m, —CH₂N—), 4.35 (2H, t, J=5.6 Hz, —OCH₂—), 7.41 (1H, dd, J=8.8, 2.8 Hz, H-2), 7.75 (1H, d, J=2.4 Hz, H-4), 7.86 (2H, m, H-6 and H-7), 8.23 (3H, m, H-1, H-5 and H-8).

¹³C NMR (CD₃OD): δ 26.0 (Me), 27.9 (—OCH₂CH₂—), 40.1 (—CH₂NH—), 57.9

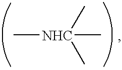

### 66.0 (—OCH₂—), 112.0 (C-4), 122.1 (C-2), 128.0 (C-5 and C-8), 128.6 (C-9a), 130.8 (C-1), 134.8 (C-8a), 134.9 (C-10a), 135.1 (C-7), 135.6 (C-6), 136.9 (C-4a), 164.8 (C-3), 183.3 (C-9), 184.2 (C-10).

EIMS (70 eV) m/z (% rel int.): 337 (1) [M]⁺.

HREIMS m/z [M]⁺ 337.1682 (calcd for C₂₁H₂₃NO₃, 337.1678).

### Synthesis Ex. 11

### 3-[3-(Cyclohexylamino)-propoxy]-9,10-anthraquin one (Compound 15)

The title compound was obtained as a pink powder (0.034 mg, 0.092 mmol, 32.0%) according to the procedures set forth in the above Synthesis Example 5, except that cyclohexylamine (1.33 g, 19.39 mmol) was used in place of diethylamine.

### Detected Properties of the Title Compound:

IR (KBr): 1675, I591 cm⁻¹.

¹H NMR (DMSO-d₆): δ 1.34 (6H, m, —CH₂CH₂CH₂—), 1.82 (2H, m, —OCH₂CH₂—), 2.15 (4H, m, —CH₂CHCH₂—), 2.82 (1H, m,

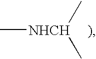

### 3.08 (2H, m, —CH₂NH—), 4.29 (2H, t, J=5.6 Hz, —OCH₂—), 7.35 (1H, dd, J=8.8, 2.8 Hz, H-2), 7.67 (1H, d, J=2.4 Hz, H-4), 7.66 (2H, m, H-6 and H-7), 8.16 (3H, m, H-1, H-5 and H-8).

¹³C NMR (DMSO-d₆): δ 25.7 (—CH₂—), 26.5 (—CH₂—), 27.9 (—CH₂—), 28.3 (—OCH₂CH₂—), 43.6 (—CH₂NH—), 58.4

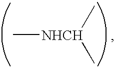

### 67.4 (—OCH₂—), 112.0 (C-4), 122.1 (C-2), 126.5 (C-9a), 126.7 (C-8), 126.7 (C-5), 129.6 (C-1), 133.0 (C-8a), 133.1 (C-10a), 134.2 (C-7), 134.7 (C-6), 135.1 (C-4a), 163.9 (C-3), 181.3 (C-9), 182.4 (C-10).

EIMS (70 eV) m/z (% rel int.): 363 (1) [M]⁺.

HREIMS m/z [M]⁺ 363.1834 (ca1cd for C₂₃H₂₅NO₃, 337.1834).

### Synthesis Ex. 12

### 3-[3-(4-Methylpiperazinopropoxy)-propoxy]-9,10-anthraquinone (Compound 16)

The title compound was obtained as a yellow powder (0.037 mg, 0.103 mmol, 35.4%) according to the procedures set forth in the above Synthesis Example 5, except that 1-methylpiperazine (1.33 g, 19.39 mmol) was used in place of diethylamine.

### Detected Properties of the Title Compound:

IR (KBr) 1672, 1594 cm⁻¹.

¹H NMR (DMSO-d₆): δ 1.92 (2H, m, —OCH₂CH₂—), 2.13 (3H, s, Me), 2.33-2.44 (10H, m, —CH₂N(CH₂CH₂)₂N—), 4.31 (2H, t, J=5.6, —OCH₂—), 7.32 (1H, dd, J=8.8, 2.8 Hz, H-2), 7.57 (1H, d, J=2.4 Hz, H-4), 7.91 (2H, m, H-6 and H-7), 8.15 (3H, m, H-1, H-5 and H-8).

¹³C NMR (DMSO-d₆): δ 26.4 (Me), 46.2 (—OCH₂CH₂—), 53.2 (—N(CH₂CH₂)₂N—), 54.6 (—CH₂N—), 55.2 (—N(CH₂CH₂)₂N—), 67.3 (—OCH₂—), 111.1 (C-4), 121.6 (C-2), 127.1 (C-9a), 127.2 (C-5 and C-8), 130.0 (C-1), 133.6 (C-8a and C-10a), 134.6 (C-6 and C-7), 135.1 (C-4a), 163.9 (C-3), 181.8 (C-9), 182.9 (C-10).

EIMS (70 eV) m/z (% rel int.): 364 (2) [M]⁺. HREIMS m/z [M]⁺ 364.1792 (ca1cd for C₂₂H₂₄N₂O₃, 364.1788).

### Pharmacological Experiments

In order to determine the biological activities of Compounds 5-16 prepared in the above examples, the following pharmacological experiments were performed.

### Experiment 1. In vitro Anticancer Assay (Cytotoxicity):

As a preliminary screening for compounds having potential to act as an anti-cancer drug, Compounds 5-16 obtained in the above synthesis examples were subjected to in vitro anti-cancer assay to determine whether or not they are capable of inhibiting the growth of any one of the selected four human tumor cells, i.e., Hep3B, HepG2, HT-29 and MCF-7.

Hep3B and HepG2 (human hepatoma), HT-29 (human colorectal adenocarcinoma) and MCF-7 (human breast adenocarcinoma) cells were obtained from American Type Culture Collection (ATCC, Rockville, Md.) and grown in Dulbecco's modified Eagle medium (DMEM; Gibco BRL, Grand Island, N.Y.)(Tsai, C.-M. et al. (1989), Cancer Res., 49: 2390-2397; Liu, H.-S. et al. (1992), Cancer Res., 52: 983-989) containing 10% of fetal bovine serum (FBS; Gibco BRL), 2 mM L-glutamine, 100 U/mL penicillin and 100 μg/mL streptomycin. For the microassay, the growth medium was supplemented with 10 mM HEPES buffer (pH 7.3) and incubated at 37° C. in a CO₂ incubator.

The cytotoxicity was determined by colorimetric MTT (3-(4,5-dimethylthiazole-2-yl)-2,5-diphenyl-1H-tetrazolium bromide)(Sigma, St. Louis, Mo.) assay as described previously (Wei, Bai-Luh et al. (2000), Eur. J. Med. Chem., 35, 1089-1098). Briefly, cells (5×10³/well) were plated in 96-well plates and incubated in medium for 6 h, followed by addition of serial dilutions (50 μL/well) of each of the tested compounds. After incubation at 37° C. for 5 or 6 days, the cells were pulsed with 10 μL of MTT (5 mg/mL) and incubated for an additional 4 h at 37° C. Reduced MTT was measured spectrophotometrically using a Dynatech MR 5000 microplate reader (Dynatech Laboratories, Va.) at 550 nm after lysis of cells with 100 μL of 10% SDS in 0.01 M HCl. Control wells contained medium plus cells (total absorbance) or medium alone (background absorbance). Cell death was calculated as the percentage of MTT inhibition:

$$\% Inhibition = \frac{1 \text{-} mean experimental absorbance}{mean control absrobance} \times 100$$

In the experiment, 5-fluorouracil(5-Fu) was used as a positive control.

### Results:

Table 1 summarizes the ED₅₀ values for the tested compounds in relation to the four different human tumor cell lines.

TABLE 1
Cytotoxicity of Compounds 5–16 (ED₅₀ values in μM)

Cell line

| Compound | HepG2 | Hep3B | HT-29 | MCF-7 |
|---|---|---|---|---|
| 5 | 1.23 | 9.09 | 56.17 | 171.95 |
| 6 | 5.52 | 5.97 | 4.72 | n.d. |
| 7 | 5.30 | 6.80 | 7.93 | n.d. |
| 8 | 4.42 | 7.93 | 6.52 | n.d. |
| 9 | 17.21 | 12.76 | 11.87 | 16.07 |
| 10 | 8.92 | 12.38 | 6.19 | 8.36 |
| 11 | 5.39 | 11.46 | 7.12 | 11.46 |
| 12 | 7.12 | 14.24 | 14.54 | 10.39 |
| 13 | 10.39 | 9.26 | 7.72 | 14.24 |
| 14 | 17.51 | 23.15 | 12.88 | 20.77 |
| 15 | 15.98 | 4.96 | 3.86 | 5.79 |
| 16 | 8.41 | 13.74 | 4.67 | 3.02 |
| 5-FU | 0.25 | 0.55 | 0.57 | |

For a significant activity of Compound 5–16, an ED₅₀ ≦ 4.0 μg/mL is required;
positive control: 5-fluorouracil (5-Fu);
n.d.: not determined.

Cytotoxic activities of a series of DHA (compound 5), MHA (compounds 6-8) and NHA (compounds 9-16) derivatives were studied against four different cancer cell types. The results are shown in Table 1.

Compound 1 failed to show cytotoxic effects against several cancer cell lines in vitro (data not shown) while prenylation at C-4 of compound 1 (i.e., compound 5) resulted in potent cytotoxicity against human HepG2 cells in vitro, indicating that a prenyl substitution at C-4 of compound 1 selectively enhanced the cytotoxic effects against HepG2 and Hep3B cells.

Compounds 2, 3 and 4 showed no significant cytotoxic activity against human HepG2, Hep3B and HT-29 cells (data not shown). However, when the bromo atom of Compound 2 or Compound 4 was replaced by an amino group (see compounds 6-16), enhanced cytotoxic effects against several different human cancer cell lines in vitro were observed.

In the applicant's earlier study, a 1-hydroxy-3-[(3-amino)propoxy]-9,10-anthraquinone (MHA) derivative, ie., 1-hydroxy-3-[3-(dimethylamino)-propoxy]-9,10-anthraquinone (Compound 19 reported in Wei, Bai-Luh et al. (2000), supra) exhibited significant cytotoxic activities against human HepG2 and Hep3B cell lines in a concentration-dependent manner with ED₅₀ values of about 2.6 and 7.1 μM, respectively.

Comparing the results of Compounds 6-8 shown in the above Table 1, it is noted that increasing the carbon number of the N-substituted alkyl side chain of Compound 6 resulted in an enhancement of the cytotoxic activity against human HepG2 cells, but a reduction of the cytotoxic activity against human Hep3B cells.

Compound 11, which has an isopropyl group substituted at N-atom of the NHA derivatives, exhibited potent cytotoxic activity against human HepG2 cells. However, increasing the carbon number of the N-substituted alkyl side chain of Compound 11 did not enhance the cytotoxic activity against human HepG2 cells (see compounds 12-14).

Compound 15, which has a cyclohexyl group substituted at N-atom of the NHA derivatives, exhibited stronger cytotoxic activities against human Hep3B, HT-29 and MCF-7 cells than those of the same series of compounds. Compound 16, which has a 4-methylpiperazyl group substituted at N-atom of the NHA derivatives, showed potent cytotoxic activity against human HT-29 and MCF-7 cells but less potent cytotoxic activity against human HepG2 and Hep3B cells. The results shown in Table 1 reveal that an N-substituted cyclic or heterocyclic side chain at the 3-position of the NHA derivatives significantly enhanced the cytotoxic activities of said derivatives against HT-29 and MCF-7 cells.

### Experiment 2. Flow Cytometry Analysis

Flow cytometry was used to determine the change of cell cycle of human cancer cells caused by the treatment of a compound according to this invention.

MCF cells (1×10⁴ cells/mL) were treated with various concentrations (6.9, 13.7, and 27.5 μM) of Compound 16 for different time periods (24 h, 48 h and 72 h), followed by washing with PBS so as to terminate the reaction. After fixation with 4% paraformaldehyde/PBS (pH 7.4) at room temperature for 30 min, the cells were centrifuged at 1,000 rpm for 10 min and then permeabilized with 0.1% Triton-X-100/0.1% sodium citrate at 4° C. for 2 min. Subsequently, propidium iodide (Sigma) in PBS (10 μg/mL) was added to stain the cells at 37° C. for 30 min. The intensity of fluorescence was measured with a FACScan flow cytometer (Becton Dickinson, Mountain View, Calif.). A minimum of 5000 cell counts was collected for the analysis by LYSIS II Software.

### Results:

It has been recognized that apoptotic cells have reduced DNA stainability with a variety of fluorochromes (Ojeda, F. et al. (1990), Cell Immunol., 125:535-539; Afanasev, V. N. et al. (1986), FEBS Lett., 194: 347-350). The appearance of cells with low DNA stainability forms a “sub-G1 peak”, which has been considered to be the hallmark of cell death by apoptosis (Darzynkiewicz, Z. et al. (1992), Cytometry, 13, 795-808). In the applicants' earlier study, it was found that the MHA derivatives induced cell death by apoptosis (Wei, Bai-Luh et al. (2000), supra). In this invention, the applicants proposed that the NHA derivatives might induce cell death by the same way. MCF-7 cells were treated with different concentrations of representative Compound 16 for different time periods.

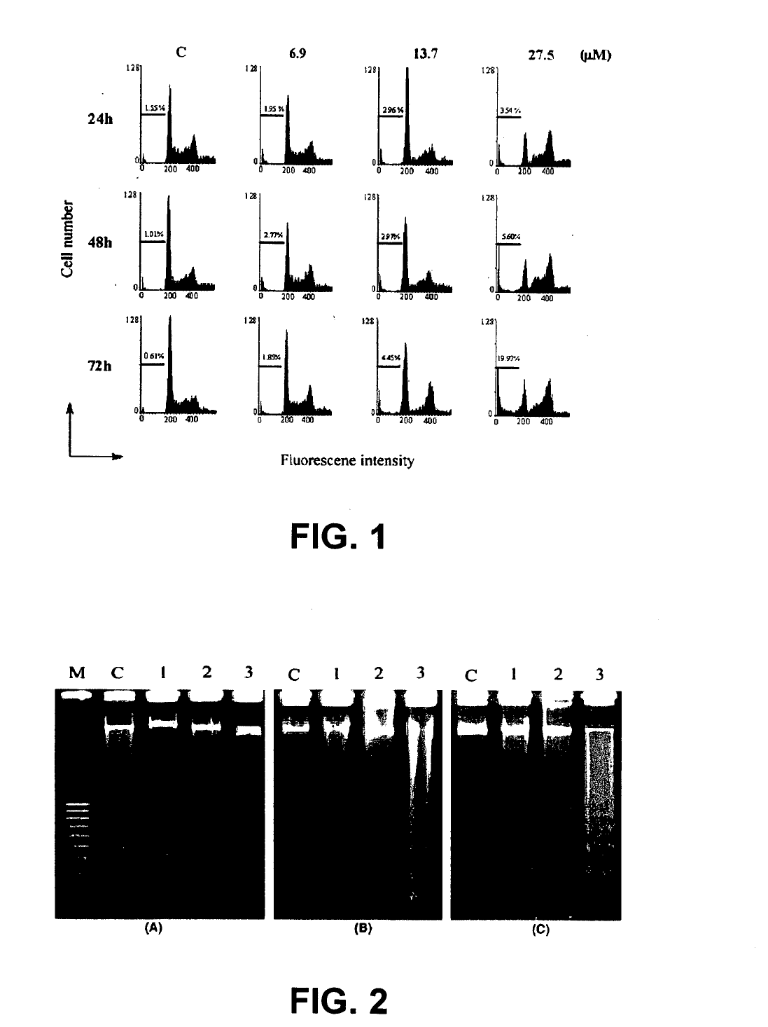

As can be seen from FIG. 1, a sub-G1 peak was detected in the DNA histograms of Compound 16 at various concentrations for different time periods. The shift of G₀/G₁ cell cycles to the G₂/M phase was increased in a dose-dependent manner in the MCF-7 cells treated with Compound 16 for different time periods. However, a maximum 19.97% apoptosis cells were detected at 72 h. The flow cytometry analysis results reveal that Compound 16 could arrest G₂/M and S phases.

### Experiment 3. DNA Fragmentation Assay

DNA fragmentation in general is used to characterize cell death by apoptosis (Wyllic, A. H. et al. (1980), Int. Rev. Cytol., 68:251-306; Arends, M. J. and Wyllic, A. H. (1991), Int. Rev. Exp. Pathol., 32:223-254). Apoptosis of MCF-7 cells after treatment with the representative Compound 16 was also studied by DNA fragmentation assay.

MCF cells (1×10⁴ cells/mL) in 150-mm plates were treated with various concentrations (6.9, 13.7, and 27.5 μM) of Compound 16 for different time periods (24 h, 48 h and 72 h), followed by washing with PBS so as to terminate the reaction. After the addition of 100 μL lysis buffer [1% of NP-40 (Sigma) in 20 mM EDTA, 50 mM Tris-HCl, pH 7.5] and mixing, the resultant cell lysates were centrifuged at 14,000 rpm for 5 min and the supernatants were collected. The supernatants were incubated with 50 μL of RNase A (20 mg/mL) and 20 μL of SDS (10%) at 56° C. for 2 h. Thereafter, 35 μL of proteinase K (20 mg/mL) was added and the resultant mixture was incubated at 37° C. overnight. DNA fragments were precipitated after the addition of 150 μL of 10M NH₄OAc and 1.2 mL of 100% ethanol at −20° C. overnight. After centrifuging and drying, the thus-obtained DNA pellets were re-suspended in 15 μL Tris-EDTA buffer and electrophoresed on a 1% agarose gel in TBE buffer at 30 V for 8 h. DNA ladder was observed after staining with ethidium bromide solution and exposure to UV light (Chang, M.-Y. et al. (1998), Biochem. Biophys. Res. Commun., 248:62-68).

### Results:

Referring to FIG. 2, DNA fragmentation in MCF-7 cells was significantly observed after 48 and 72 h incubation with Compound 16 (6.9, 13.7 and 27.5 μM), suggesting that Compound 16 might induce the shift of G₀/G₁ phase to G₂/M and S phases and caused cell death by apoptosis.

In conclusion, almost all compounds have potent inhibitory activity against HepG2, Hep3B and HT-29 cell lines in vitro. Compound 5 exhibited selective cytotoxicity against HepG2 in a concentration-dependent manner with ED50 value of 1.23±0.05 μM. Therefore, it is contemplated that compound 5 may serve as a lead structure in the design and synthesis of a new series of 1,3-dihydroxyl-9,10-anthraquinone derivatives. A sub-G1 cell stage and DNA fragmentation in MCF-7 cells were significantly observed after 48 h incubation with Compound 16. It is predicted that the compounds as prepared from the above synthesis examples may induce cell death by apoptosis and they might be developed as anti-cancer agents. Further experiments are needed to elucidate the mechanism of action of Compounds 5 and 16.

All patents and literature references cited in the present specification are hereby incorporated by reference in their entirety. In case of conflict, the present description, including definitions, will prevail.

While the invention has been described with reference to the above specific embodiments, it is apparent that numerous modifications and variations can be made without departing from the scope and spirit of this invention. It is therefore intended that this invention be limited only as indicated by the appended claims.

What is claimed is:

1. A compound of formula (A): 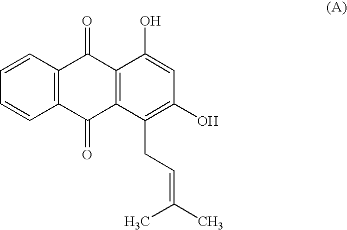
or a pharmaceutically acceptable salt thereof.

2. A pharmaceutical composition comprising a compound of formula (A) as claimed in claim 1, or a pharmaceutically acceptable salt thereof, and, optionally, a pharmaceutically acceptable carrier.

3. A method for inhibiting the growth of a tumor/cancer cell in a subject, comprising administering to the subject a therapeutically effective amount of a compound of formula (A): 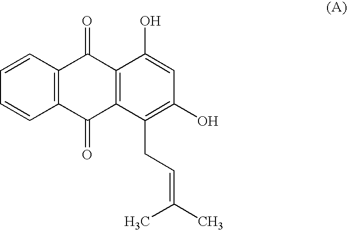
or a pharmaceutically acceptable salt or solvate thereof.

4. The method of claim 3, wherein the tumor/cancer cell is selected from the group consisting of human liver cancer cells, human colon/rectal cancer cells, and human breast cancer cells.

5. The method of claim 3, wherein administering the compound of formula (A), or a pharmaceutically acceptable salt thereof, results in the growth inhibition of said tumor/cancer cell by apoptosis.

6. A compound of formula (B): 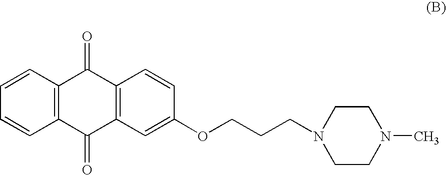
or a pharmaceutically acceptable salt thereof.

7. A pharmaceutical composition comprising a compound of formula (B) as claimed in claim 6, or a pharmaceutically acceptable salt thereof, and, optionally, a pharmaceutically acceptable carrier.

8. A method for inhibiting the growth of a tumor/cancer cell in a subject, comprising administering to the subject a therapeutically effective amount of a compound of formula (B): 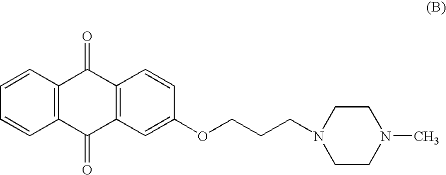
or a pharmaceutically acceptable salt or solvate thereof.

9. The method of claim 8, wherein the tumor/cancer cell is selected from the group consisting of human liver cancer cells, human colon/rectal cancer cells, and human breast cancer cells.

10. The method of claim 8, wherein administering the compound of formula (B), or a pharmaceutically acceptable salt thereof, results in the growth inhibition of said tumor/cancer cell by apoptosis.
